(19) United States
(12) Patent Application Publication
(10) Pub. No.: US 2026/0174519 A1
(43) Pub. Date: Jun. 25, 2026

(54) SYSTEM FOR OPTIMIZING A CONTROL POINT OF A ROBOTICALLY CONTROLLABLE INSTRUMENT

(71) Applicant: KARL STORZ SE & Co. KG, Tuttlingen (DE)

(72) Inventors: Shashank SHARMA, Tuttlingen (DE); Sreekanth KANA, Tuttlingen (DE)

(73) Assignee: KARL STORZ SE & Co. KG, Tuttlingen (DE)

(21) Appl. No.: 19/420,904

(22) Filed: Dec. 16, 2025

(30) Foreign Application Priority Data
Dec. 19, 2024 (DE) 10 2024 138 989.3

(51) Int. Cl. A61B 34/37; A61B 1/00

(52) U.S. Cl. CPC A61B 34/37; A61B 1/00006; A61B 1/0004; A61B 1/00087; A61B 1/00149

(57) ABSTRACT

A system and a method for dynamically optimizing a control point of at least one robotically controllable instrument are provided. The system comprises: a recording device configured to continuously record information about the position and a control point of the at least one robotically controllable instrument; an identification device configured to identify at least one optimized control point on the basis of the information relating to the position and control point of the at least one robotically controllable instrument; and a control device configured to transmit motion commands to a robot controlling the at least one robotically controllable instrument, wherein the motion commands are designed to instruct the robot to control the at least one robotically controllable instrument from one of the at least one optimized control points.

### CROSS-REFERENCE TO RELATED APPLICATIONS

This application claims priority of German Patent Application No. DE 10 2024 138 989.3 filed on Dec. 19, 2024, the contents of which are incorporated herein.

### TECHNICAL FIELD

The present disclosure relates to a system and to a method for dynamically optimizing a control point of at least one robotically controllable instrument, particularly in the medical field. The present disclosure further relates to a master-slave system for performing medical procedures.

### BACKGROUND

In medical procedures and surgeries, surgeons/operators are usually supported by partially or fully robotic systems. This applies in particular to robot-assisted surgery, in which one or more robots are used to handle various medical instruments and equipment, which robots are monitored, programmed, or remotely controlled by a surgeon/operator. The use of robotic components in the medical field leads to an increase in accuracy and thus makes the surgeon's work easier.

When remotely controlling robotic systems, especially master-slave systems, attempts are made to make the surgeon's motion commands as ergonomic as possible. Important parameters here are the mobility of the robot arm and the control point, from where the robot holds the medical instrument or equipment, that is chosen.

Currently, most robotic systems in robot-assisted surgery operate with a control point for an instrument that is set prior to a medical procedure.

However, during a medical procedure, certain movements of an instrument from the predetermined control point are not ergonomic. The surgeon may not be familiar with, or used to, the resulting movements performed by the robot with the instrument. This could lead to the remote control of the robot not being perceived as intuitive. This sometimes presents a limitation and makes the surgeon's work more difficult.

It is therefore important to provide systems and methods that make the remote control of robots in master-slave systems intuitive and ergonomic for the surgeon.

### SUMMARY

It is therefore an object of the present disclosure to provide a system and a method that enable improved remote control of a robot controlling at least one instrument.

This object is achieved by the subject matter of the independent claims of the present disclosure.

According to a first aspect, a system for dynamically optimizing a control point of at least one robotically controllable instrument is provided, comprising: a recording device configured to continuously record information relating to the position and a control point of the at least one robotically controllable instrument; an identification device configured to identify at least one optimized control point on the basis of the information relating to the position and control point of the at least one robotically controllable instrument; and a control device configured to transmit motion commands to a robot controlling the at least one robotically controllable instrument, wherein the motion commands are designed to instruct the robot to control, in particular to move, the at least one robotically controllable instrument from one of the at least one optimized control points.

A fundamental concept of the present disclosure is to be able to dynamically (i.e., continuously or uninterruptedly) adapt the control point of a robot-controlled instrument during a procedure. The system according to the disclosure is designed to identify new control points on the basis of at least one existing control point and the position (or orientation) of the instrument, wherein the new control points can be used during the procedure.

During a medical procedure using hand-held instruments, the way in which a surgeon holds his instruments constantly changes. The surgeon thus attempts to find the optimal way of holding the instrument, which depends, among other things, upon the task to be performed during the different phases of the operation. In robot-assisted surgeries, the surgeon does not hold the instrument himself, but, rather, by means of a robotic arm, which is controlled by the surgeon via an operating unit. The operating unit can control the robot (and thus the instrument) via a joystick and/or voice commands. Surgeons have developed their habits and skills through operations using hand-held instruments. With most conventional robotic systems, it is difficult to maintain these habits or to fully deploy the skills, since the instrument is not controlled directly by the surgeon, but indirectly via the robot.

One advantage of the present disclosure is that, with the system according to the disclosure, the control point of an instrument is no longer static, but can be dynamically changed, in particular automatically. This makes the movements of a robot in master-slave systems more closely resemble those of a surgeon. The optimized control points are automatically selected.

In this way, the system mimics the movements of a surgeon in a more realistic way so that the surgeon can perceive the movements that are or are to be performed by the robot as his own. The ability to adapt the control point during a medical procedure gives the surgeon flexibility when interacting with the instrument and allows him to choose the most effective control strategy for each specific surgical task. For example, when sewing, precise control of the instrument tip is crucial for accuracy. Conversely, defining the control point at the wrist of the instrument can be advantageous in scenarios such as the preparation of complex operations, where fine-tuning the wrist alignment ensures that the instrument is optimally aligned before focusing on the movement of the tip.

Advantageously, the system according to the first aspect of the disclosure can be used for all types of robot-assisted procedures (including medical procedures) and for all types of instruments.

A robotically controllable instrument is understood here to mean an instrument or equipment that can be operated by the use of a robot. The robot can be a robot with a multi-joint robot arm that has an instrument holder at its distal end.

A control point is a location on an instrument or equipment that serves as a reference point or starting point for transferring the surgeon's movement from the operating apparatus to the instrument. A control point can be used by a robot as a stopping point (i.e., a fixed point) to control, in particular move, the instrument therefrom. In other words, a control point is a point on which motion control (the surgeon's hand movements, which are transmitted by the robot) focuses (and in some cases is limited to).

Information about a position of an instrument usually at least includes information about the location of at least some of the instrument's boundaries and/or their orientation. For example, coordinates of a reference system can be assigned to the object boundaries (e.g., through image registration). Determining the position of an instrument is understood in particular to mean identifying its spatial coordinates.

Motion commands contain information for the robot (preferably in machine language) so that the robot can change the point where an instrument is held (from an existing control point to another control point).

Although some functions are described above and below as being performed by “devices” or “interfaces,” it goes without saying that this does not necessarily mean that such devices or interfaces are provided as separate apparatuses. In cases in which one or more devices or interfaces are wholly or partially provided as software, the devices or interfaces can be implemented by program code portions or snippets, which differ from one another, but can also be interlinked.

Similarly, in the case in which one or more devices or interfaces are provided as hardware, the functions of one or more devices or interfaces may be provided by the same hardware component, or the functions of one device or one interface or the functions of a plurality of devices or interfaces may be distributed among a plurality of hardware components that do not necessarily correspond one-to-one to the devices or interfaces. For this reason, any apparatus, system, method, etc., that has all the features and functions attributed to a particular device and/or a particular interface is to be understood as forming, comprising, or implementing the device and/or interface.

In particular, there is the possibility that some or all of the devices and interfaces will be implemented by executable program code.

In particular, the recording device and/or the identification device and/or the control device can be implemented as any computing apparatus or means, especially for executing software, an app, or an algorithm. For example, the recording device and/or the identification device and/or the control device can comprise at least one processor, such as at least one central processor, CPU, and/or at least one graphics processor, GPU, and/or at least one field-programmable gate array, FPGA, and/or at least one application-specific integrated circuit, ASIC, and/or any combination of the above. The recording device and/or the identification device and/or the control device can further comprise a random access memory operatively connected to the at least one processor, and/or a non-volatile memory operatively connected to the at least one processor and/or to the random access memory. The recording device and/or the identification device and/or the control device may be partially and/or completely implemented in a local apparatus (such as in an operating unit or in an operator console of a robot-assisted surgical system) and/or partially and/or completely implemented in a remote system, such as through a cloud-computing platform. The identification device can in particular be equipped with machine learning models.

The recording device can be configured to record the position and control point of the instrument by processing image data from one or more imaging devices (e.g., the image stream of an endoscope), in particular in real time, or alternatively also from a picture archiving and communications system (PACS), wherein the latter is also performable in real time, i.e., in particular as soon as the image data is received in the PACS.

Further advantages of the disclosure will be explained below with reference to the subject matter of the dependent claims and in particular with reference to the description of the figures.

According to some preferred embodiments, variants, or refinements of embodiments, the robotically controllable instrument is a medical instrument, in particular for use in minimally invasive endoscopic procedures. Although the principles of the disclosure are applicable to any master-slave systems, applications in the medical field, in particular robot-assisted, minimally invasive procedures, are preferred. Examples of robot-controlled instruments in this connection include tweezers, pliers, needle holders, or staplers.

According to some preferred embodiments, variants, or refinements of embodiments, the identification device is further configured to identify an optimized control region. Instead of identifying just one or more control points, the identification device can offer an entire region. This serves to make the selection of optimized control points more flexible, in order to better accommodate the habits of different operators. The optimized control region can, for example, be the jaw part of pliers or forceps. In these embodiments, an operator who wishes to control the jaw part, e.g., from a control point that is as far away as possible, can select his personal optimal control point.

According to some preferred embodiments, variants, or refinements of embodiments, the system according to the disclosure further comprises a user interface designed to display a plurality of control points and to transmit to the control device a control point then selected by a user (or operator) from the plurality of control points as the optimized control point. In some embodiments of the disclosure, it is provided that the identification device identify more than one optimized control point.

The user interface allows the operator, for example, to select a new control point from the plurality of control points. The user interface can preferably be designed to display the current (existing) control point together with the at least one control point identified. The control points can in particular be color-coded, number-coded, letter-coded, etc., wherein the coding corresponds to a list of recommendations ranked by the identification device.

According to some preferred embodiments, variants, or refinements of embodiments, the user interface comprises a display and a controller, wherein the display is configured to display a plurality of control points, and wherein the controller is configured to allow a user (or operator) to thus select the desired control point from the plurality of control points. The display can be designed to superimpose the plurality of control points on an image of the instrument. The operator can then navigate through the instrument's image using the controller, e.g., with a cursor, and graphically select an optimized control point using the cursor.

As explained above, the system according to the disclosure can also graphically represent optimized control regions. Control points, e.g., within the jaw part (as an example of a control region), can be selected using the controller. In medical procedures where a plurality of instruments are controlled by a robot, the user interface may also include a mechanism by which the operator can first select one instrument from the plurality of instruments. This can be displayed, for example, in a menu on the display.

According to some preferred embodiments, variants, or refinements of embodiments, the identification device is further configured to select the optimized control point from a list of predetermined control points, wherein the list of predetermined control points is dependent upon the robotically controllable instrument. In these embodiments of the disclosure, the control points are therefore limited to the specified list. This conserves computing resources. For example, the list of control points for forceps could include a control point at a joint, a control point at a jaw part, and a control point at a tip. The identification device can thus determine which of these three control points is the optimized control point during a medical procedure.

The number of specified control points and their position depend upon the instrument. For example, a complex instrument may have a plurality of specified control points.

According to some preferred embodiments, variants, or refinements of embodiments, the identification device further comprises an artificial intelligence entity equipped with one or more machine learning models and trained and designed to automatically select an optimized control point. The algorithms of the identification device can be supported, at least partially, by methods of artificial intelligence-for example, by being fed thereby or providing input data therefor.

The machine learning model(s) may also include deep learning models that implement one or more artificial neural networks. Artificial neural networks are to be understood here as models with any architecture and any number of intermediate layers, such as Convolutional Neural Networks (CNN's). The identification of optimized control points can be performed using a typical artificial neural network from the prior art-for example, a ResNet50 trained with data from medical procedures in which the instruments are handled by surgeons.

According to some preferred embodiments, variants, or refinements of embodiments, the artificial intelligence entity is designed to automatically select the optimized control point on the basis of information relating to at least one robotically controllable instrument to be used and/or relative to a user (e.g., an operator) and/or a procedure to be performed. The optimal control points are preferably identified using as much relevant information as possible. The optimized control points can therefore be determined taking into account the instrument and the type of procedure. The control points can also be individually adapted to each operator. Specifically, different operators sometimes handle the instruments according to different preferences. The artificial intelligence entity can adapt the control points to these operator preferences.

According to some preferred embodiments, variants, or refinements of embodiments, the control points identified can be stored in a database for an operator. In this way, the identification device can retrieve the control points preferred by an operator and accordingly determine the identified control points by taking into account the surgeon's preferences.

According to some preferred embodiments, variants, or refinements of embodiments, the artificial intelligence entity is designed to determine the optimized control point using information from at least one optical sensor. Furthermore, the at least one optical sensor (which may be part of the system or separate therefrom) is designed to capture images at least of the robotically controllable instrument and the control point. Image data from an optical sensor (e.g., the camera of an endoscope) can be used to refine the position of the control points. In these embodiments, the artificial intelligence entity, AIE, is preferably equipped with image processing software to enable or refine the recognition of the instrument.

For example, the AIE can automatically identify tissue near the instrument and automatically identify the optimized control point on the basis of the position of the tissue. In some embodiments, a property of the tissue, such as a tissue type or the like, can also be automatically identified using the AIE, and the identification of the optimized control point can be based thereon, among other things.

According to some preferred embodiments, variants, or refinements of embodiments, the artificial intelligence entity is further configured to identify the optimized control point in accordance with operation planning (OP planning). In this connection, operation planning is to be understood as meaning a compilation of information that describes the course of the medical procedure. The artificial intelligence entity can, for example, be equipped with generative artificial intelligence methods so that it can determine the optimized control points on the basis of the operation planning instructions. In these embodiments, the identification device can further be configured to receive such operation planning-for example, from a local user interface or from a central computing device.

According to some preferred embodiments, variants, or refinements of embodiments, the system according to the disclosure further comprises at least one force sensor designed to record information about the force that is or can be exerted by the robotically controllable instrument and to transmit the recorded information to the identification device. Information about a force that is exerted is relevant, for example, to finding a better control point, e.g., a control point from which forces can be transferred more efficiently. The identification of the at least one optimized control point can therefore optionally be based, among other things, upon the information about the exerted or exertable force; for example, it can be done in such a way that the maximum force can be exerted, or in such a way that particularly precise amounts of force can be exerted.

According to a second aspect, the present disclosure provides a computer-implemented method for dynamically optimizing the control point of at least one robotically controllable instrument, at least comprising the steps of: continuously recording information about the position and a control point of at least one robotically controllable instrument; identifying at least one optimized control point on the basis of the information about the position and control point of the at least one robotically controllable instrument; and transmitting motion commands to a robot that controls the at least one robotically controllable instrument, wherein the motion commands are designed to instruct the robot to control the at least one robotically controllable instrument from one of the at least one optimized control points.

The computer-implemented method of the second aspect can preferably be carried out using the system according to the first aspect. Similarly, the various embodiments of the system can be operated according to embodiments of the computer-implemented method of the second aspect. The method can therefore be adapted according to all options, variants, embodiments, and refinements described in relation to the system according to the disclosure, and vice versa.

According to a third aspect, the present disclosure provides a master-slave system, at least comprising: a robotically controllable instrument; a robot configured to control the robotically controllable instrument from a control point; and a system according to the first aspect of the disclosure, wherein the system is configured to identify at least one optimized control point and to transmit motion commands to the robot, wherein the motion commands are configured to instruct the robot to control the at least one robotically controllable instrument from one of the at least one optimized control points.

According to a fourth aspect, the disclosure provides a computer program product that comprises executable program code that is configured to carry out the method according to an embodiment of the second aspect of the present disclosure when executed by a computing device.

According to a fifth aspect, the disclosure provides a non-volatile, computer-readable data storage medium that comprises executable program code that is configured to carry out the method according to an embodiment of the second aspect of the present disclosure when executed by a computing device.

The non-volatile, computer-readable data storage medium can comprise or consist of any type of computer memory, in particular semiconductor memory, such as, for example, a solid-state memory. The data carrier can also comprise or consist of a CD, a DVD, a Blu-ray disc, a USB memory stick, or the like.

According to a sixth aspect, the disclosure provides a data stream comprising executable program code or configured to generate executable program code, which is configured to carry out the method according to an embodiment of the second aspect of the present disclosure when executed by a computing device.

Further advantageous variants, options, embodiments, and modifications can be found in the following figures, the detailed description, and the claims. However, it is self-evident that the detailed description and specific examples, while indicating preferred embodiments of the disclosure, are presented for illustration only, since various changes and modifications within the scope of the disclosure are apparent to a person skilled in the art.

### BRIEF DESCRIPTION OF THE FIGURES

Individual embodiments of the present disclosure will be explained in detail with reference to the following figures. The components in the drawings are not necessarily to scale, but serve to illustrate the principles of the present disclosure. Parts in the different figures that correspond to the same elements or method steps have been given the same reference signs in the figures. The numbering of method steps initially serves only to distinguish them and does not necessarily imply a corresponding order, although one variant is to carry out the steps in the order in which they are numbered. Multiple steps can also be carried out in an overlapping or simultaneous manner. In the figures:

FIG. 1 is a schematic block diagram illustrating a system according to an embodiment of the first aspect of the present disclosure;

FIG. 2 is a schematic representation of possible control points and control regions identified using the system according to the first aspect of the present disclosure;

FIG. 3 is a schematic representation illustrating a possible application for a medical procedure according to an embodiment of the present disclosure;

FIG. 4 is a schematic representation illustrating another possible application for a medical procedure according to an embodiment of the present disclosure;

FIG. 5 is a schematic block diagram illustrating a master-slave system according to an embodiment of the third aspect of the present disclosure;

FIG. 6 is a schematic flowchart illustrating a computer-implemented method according to an embodiment of the second aspect of the present disclosure;

FIG. 7 is a schematic block diagram for a computer program product according to an embodiment of the fourth aspect of the present disclosure; and

FIG. 8 is a schematic block diagram for a non-volatile, computer-readable data storage medium according to an embodiment of the fifth aspect of the present disclosure.

### DETAILED DESCRIPTION

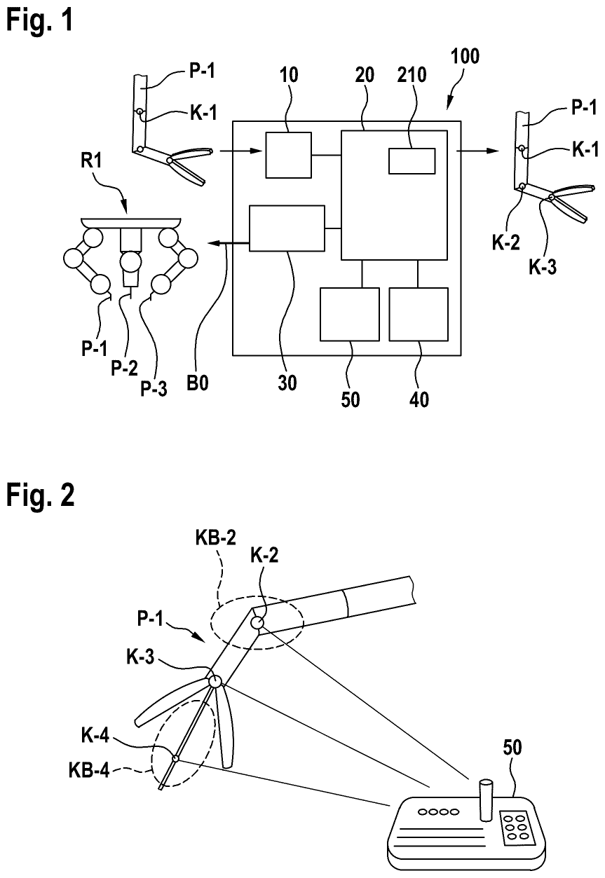

FIG. 1 shows a schematic block diagram to explain a system 100 for the dynamic optimization of a control point K-1, K-2, K-3 (hereafter partially referred to collectively as K-i) of at least one robotically controllable instrument P-1. The system 100 according to the disclosure, shown in FIG. 1, comprises a recording device 10, an identification device 20, a control device 30, a database 40, and a user interface 50.

The recording device 10 is designed to continuously record information about the position and a control point of the at least one robotically controllable instrument P-1. FIG. 1 shows, as an example of a robotically controllable instrument P-1, forceps that can be used in a minimally invasive endoscopic procedure. These forceps are controlled by a robot R1, which may have one or more multi-joint robot arms.

In the embodiment shown by way of example in FIG. 1, the robot R1 has, for example, three multi-joint robot arms. At its distal end, each multi-joint robot arm has an instrument holder which can hold the robotically controllable instruments P-1 to P-3 (hereafter sometimes collectively referred to as P-i). The robotically controllable instrument P-1 is held by the robot R1 at a control point K-1, for example, which is located at a joint of the robotically controllable instrument P-1. Other examples of a robotically controllable instrument P-i could be pliers, a needle holder, or staplers.

The recording device 10 can be configured to record the position and control point K-1 (i.e., the existing or current control point K-1) of the instrument P-1 by processing (preferably using image processing software) image data from one or more imaging devices (e.g., the image stream of an endoscope), in particular in real time, or alternatively also from a picture archiving and communications system, wherein the latter also is possible in real time.

The identification device 20 is designed to identify at least one optimized control point K-i on the basis of the information about the position and the existing control point K-1 of the robotically controllable instrument P-1. In FIG. 1, for example, an optimized control point K-2 and an optimized control point K-3 are shown on the right. It is conceivable that the optimized control point K-2 be identified at one time (with the robotically controllable instrument P-1 in a first position and with a control point K-1), and the optimized control point K-3 be identified at a later time (with the robotically controllable instrument P-1 in a second position and with a control point K-2). However, it is also possible that the identification device 20 simultaneously offer the control points K-2 and K-3 as optimized control points K-i. The control points K-2 and K-3 can, for example, be offered as equivalent alternatives, or according to a predetermined or dynamically calculated recommendation sequence.

In some embodiments of the disclosure, the optimized control points K-i can belong to a list of predetermined control points. This list of control points K-i can be stored in the database 40 and retrieved by the identification device 20. In these embodiments of the disclosure, the computational demands of the identification device 20 can be kept relatively low. In the embodiment of the disclosure shown in FIG. 1, the control points K-i could be the predetermined control points K-i for forceps. The control points K-i identified or identifiable by the identification device 20 are then limited to the control points K-1 to K-3. For example, the identification device 20 may identify the sequence of optimized control points K1-K3-K2-K1 during a medical procedure.

In some embodiments of the disclosure, the identification device 20 is further configured to identify an optimized control region KB-i. In FIG. 2, for example, three optimized control points K-2, K-3, and K-4 can be seen. Depending upon this, the robotically controllable instrument P-1 from FIG. 1 can be controlled by a robot R1 via the joint, the jaw, or the tip as the control point K-i. The optimized control point K-2 is located within an optimized control region KB-2. Accordingly, an operator can select a control point K-i within the optimized control region KB-2 to find a control point that best suits his habits. For example, an operator may prefer to hold a joint in the middle, from behind, or from the front, i.e., to select a control point at a joint, distal therefrom, or proximal thereto. The same applies to the optimized control point KB-4. An operator can select a control point K-i within the control region KB-4, including the control point K-4.

The identification device 20 can be any computing apparatus or means, in particular for executing software, an app, or an algorithm. The identification device 20 may comprise an artificial intelligence entity, AIE, 210, equipped with machine learning models. The algorithms of the identification device 20 can therefore be at least partially supported or fed by methods of artificial intelligence, or vice versa. These models are trained and designed to automatically select the optimized control point(s) K-i.

The AIE 210 can determine the optimized control point K-i, taking into account each robotically controllable instrument P-1 and the type of procedure. In particular, the optimized control point K-i can be identified using operation planning (OP planning), which describes the course of a medical procedure. The AIE 210 can, for example, be equipped with generative artificial intelligence methods so that it can determine the optimal control points (K-i) on the basis of the instructions of the operation planning. Such operation planning can be received, for example, from a local user interface or from a central computing device (not shown in FIG. 1) by the identification device 20.

The AIE 210 can also be designed to adapt the control points K-i individually for each operator on the basis of an operator's predefined preferences.

The optimized control points K-i could also be determined using information from at least one optical sensor or force sensor. Image data from an optical sensor (e.g., the camera of an endoscope) can be used to refine the position of the control points. In these embodiments, the artificial intelligence entity 210 is preferably equipped with image processing software to enable detection of the instrument P-i. Information about a force that is exerted is also relevant to finding a better control point, e.g., a control point K-i from which the forces can be transferred more efficiently or better controlled.

The control device 30 is configured to transmit motion commands B0 to the robot R1, wherein the motion commands B0 contain information (preferably in machine language) such that the robot R1 changes the point where an instrument is held (with reference to FIG. 1, from an existing control point K-1 to another control point K-2 or K-3). The control device 30 can be coupled to actuators of the robot R1 either via wires or wirelessly.

The user interface 50 is designed to display the optimized control points K-i and to transmit to the control device 30 a control point K-i selected by a user (or operator) as the optimized control point K-i. The user interface 50 can preferably be designed to display the current (existing) control point K-1 together with the at least one control point K-2 and K-3 identified.

The system 100 shown in FIG. 1 also comprises a database 40. In the database 40, the control points K-i identified can be stored for an operator in a file as a personal configuration profile. In this way, the identification device 20 can retrieve the control points K-i preferred by an operator and accordingly determine the control points K-i to be identified, while taking into account the surgeon's preferences. The database 40 can also contain a list of predetermined control points K-i for an instrument P-i.

The system 100 according to the disclosure thus makes it possible to dynamically (i.e., continuously or uninterruptedly) automatically adapt the control point K-i of a robot-controlled instrument P-i during a procedure. This makes the movements of a robot R1 in master-slave systems more consistent with those of a surgeon.

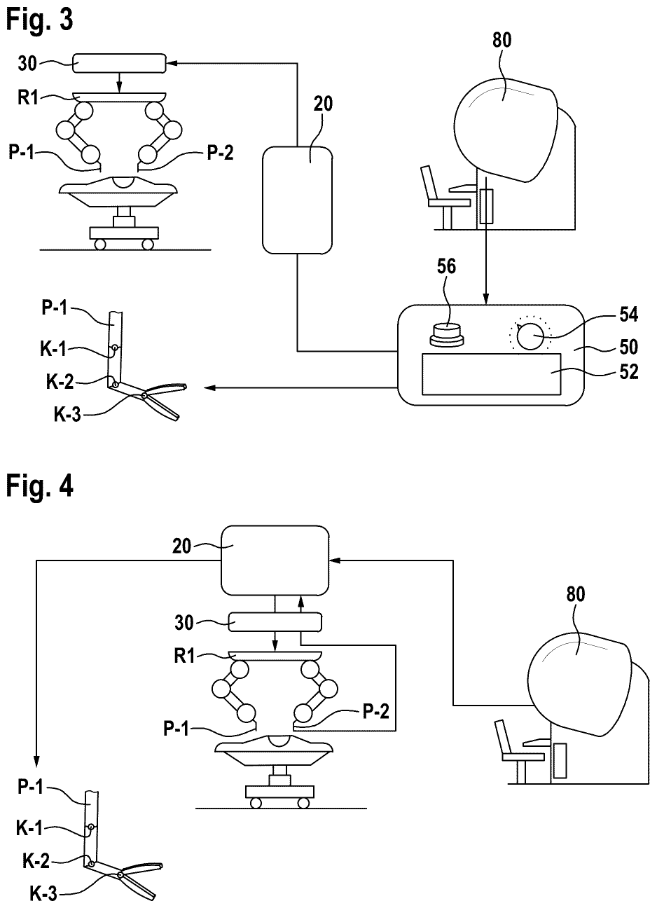

FIG. 3 shows a schematic representation illustrating a possible application for a medical procedure according to an embodiment of the present disclosure.

FIG. 3 shows a user interface 50 that can be operated by an operator. The operator can control the robot R1 using a console 80. In some embodiments, the user interface 50 can be integrated into the console 80, or vice versa.

The user interface 50 at least comprises, for example, a display 52 and a controller 54. In medical procedures where a plurality of instruments P-i are controlled by a robot R1, the user interface 50 may also include a mechanism by which the operator can first select an instrument P-i from the plurality of instruments P-i. In FIG. 3, the user interface 50 comprises a button 56 for this purpose. For example, the plurality of instruments P-i can be displayed in a menu on the display 52, from which one is selected using the button 56.

The display 54 then shows the control points K-i (preferably the current control point K-i together with the control points K-i identified by the identification device 20) and/or the identified control regions KB-i for the chosen instrument P-i, preferably superimposed on an image of the instrument P-i. For example, the operator can then navigate through the image of the instrument P-i using the controller 56, e.g., with a cursor, and select the desired control point K-i from the plurality of control points K-i.

In the embodiment of the disclosure shown in FIG. 3, the selection of the control point K-i is left to the operator. According to these embodiments, it is also provided for the operator to select a desired control point K-i, using the controller, which is not among the control points K-i identified by the identification device 20. The selection is then transmitted to the control device 30, which generates corresponding motion commands to control the robot R1 in order to change the control point K-i according to the operator's selection.

FIG. 4 shows a schematic representation illustrating another possible application for a medical procedure according to an embodiment of the present disclosure.

The embodiment of the disclosure shown in FIG. 4 constitutes an alternative to FIG. 3. In the variant according to FIG. 4, the control point K-i is automatically selected by the system 100. In this embodiment, the identification device 20 is configured to identify and select the control point K-i. The identification device 20, in particular the AIE 210, continuously records the position of the instrument P-i and, together with the current control point K-i, identifies at least one optimal control point K-i. A control point K-i is automatically selected from the at least one optimal control point K-i. The selection is used by the control device 30 to generate motion commands so that the robot controls the instrument P-1 from the selected control point K-i.

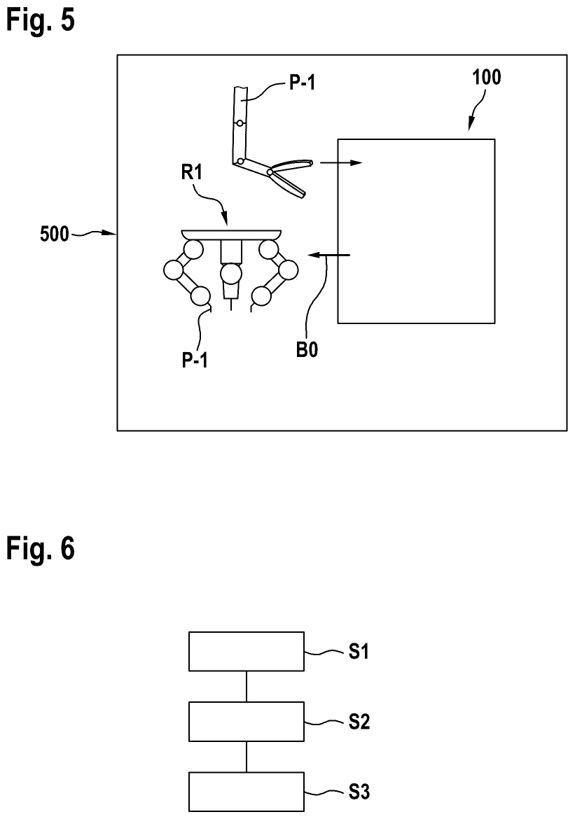

FIG. 5 is a schematic block diagram illustrating a master-slave system 500 according to an embodiment of the third aspect of the present disclosure. The master-slave system 500 comprises at least one robotically controllable instrument P-1; a robot R1 configured to control the at least one robotically controllable instrument P-1 from a control point K-i; and a system 100 according to the first aspect of the disclosure.

The system 100 is configured to identify at least one optimized control point K-i and to transmit corresponding motion commands B0 to the robot R1, wherein the motion commands B0 are designed to instruct the robot R1 to control the at least one robotically controllable instrument P-1 from one of the at least one optimized control points K-i.

FIG. 6 shows a schematic flowchart illustrating a method according to an embodiment of the second aspect of the present disclosure, i.e., a computer-implemented method for dynamically optimizing the control point K-i of at least one robotically controllable instrument P-i. The method according to FIG. 6 can be carried out in particular by means of the system 100 from FIG. 1 and can therefore be adapted according to all the options or variants described with respect to the system 100 according to the disclosure, and vice versa.

In a step S1, information about the position and a control point K-i of at least one robotically controllable instrument P-i is continuously recorded, as described above with respect to the recording device 10, for example.

In a step S2, at least one optimized control point K-i is identified on the basis of the information about the position and control point K-i of the at least one robotically controllable instrument P-i, as described above with respect to the identification device 20, for example.

In a further step S3, motion commands B0 are transmitted to a robot R1, which controls the at least one robotically controllable instrument P-i. The motion commands B0 are designed to instruct the robot R1 to control the at least one robotically controllable instrument P-i from one of the at least one optimized control points K-i, as described above with respect to the control device 20, for example.

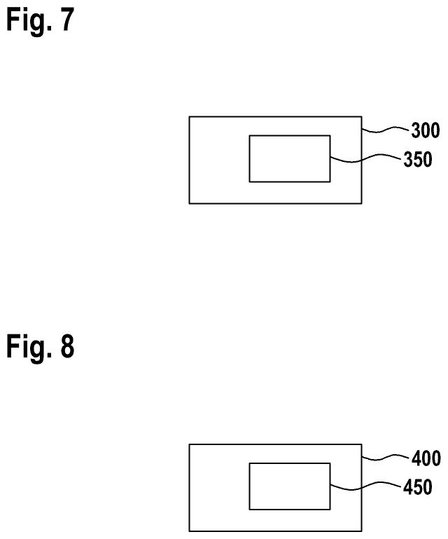

FIG. 7 shows a schematic block diagram for a computer program product 300 according to an embodiment of the third aspect of the present disclosure. The computer program product 300 comprises executable program code 350 that is designed, when executed (e.g., by a computing device), to carry out the method according to an embodiment of the present disclosure-for example, according to FIG. 6.

FIG. 8 shows a schematic block diagram for a non-volatile, computer-readable data storage medium 400 according to an embodiment of the present disclosure. The data storage medium 400 comprises executable program code 450 that is designed, when executed (e.g., by a computing device), to carry out the method according to an embodiment of the second aspect of the present disclosure-for example, according to FIG. 6.

The non-volatile, computer-readable data storage medium 400 can, for example, be designed as or comprise a semiconductor memory, e.g., an SSD memory chip. The data storage medium 400 can also have or comprise a CD, DVD, Blu-ray, or magnetic storage apparatus.

The above description of the disclosed embodiments contains only examples of possible implementations, which are described to allow a person skilled in the art to make or use the present disclosure. Various variations and modifications of these embodiments will be readily apparent to a person skilled in the art after having knowledge of the present disclosure, and the general principles defined herein can be applied to other embodiments without departing from the scope of the present disclosure.

Thus, the present disclosure is not intended to be limited to the specific embodiments shown herein, but is to be accorded the broadest scope consistent with the principles and features disclosed herein.

1. A system for dynamically optimizing a control point of at least one robotically controllable instrument, comprising:
a recording device which is configured to continuously record information about the position and a control point of the at least one robotically controllable instrument;
an identification device which is configured to identify at least one optimized control point on the basis of the information relating to the position and control point of the at least one robotically controllable instrument; and
a control device which is configured to transmit motion commands to a robot controlling the at least one robotically controllable instrument, wherein the motion commands are designed to instruct the robot to control the at least one robotically controllable instrument from one of the at least one optimized control points.

2. The system according to claim 1, wherein the robotically controllable instrument is a medical instrument, in particular for use in minimally invasive endoscopic procedures.

3. The system according to claim 1, wherein the identification device is further designed to identify an optimized control region.

4. The system according to claim 1, further comprising a user interface designed to display a plurality of control points and to transmit to the control device a control point selected by a user from the plurality of control points as the optimized control point.

5. The system according to claim 4, wherein the user interface comprises a display and a controller, wherein the display is designed to display a plurality of control points is designed to allow a user to therefore select the desired control point from the plurality of control points.

6. The system according to claim 1, wherein the identification device is further configured to select the optimized control point from a list of predetermined control points, wherein the list of predetermined control points is dependent upon the robotically controllable instrument.

7. The system according to claim 1, wherein the identification device further comprises an artificial intelligence entity equipped with one or more machine learning models and trained and designed to automatically select an optimized control point.

8. The system according to claim 7, wherein the artificial intelligence entity is designed to automatically select the optimized control point on the basis of information relating to at least one robotically controllable instrument to be used and/or relating to a user and/or a procedure to be performed.

9. The system according to claim 7, wherein the artificial intelligence entity is designed to determine the optimized control point using information from at least one optical sensor, and
wherein the at least one optical sensor is configured to capture images of at least the robotically controllable instrument and the control point.

10. The system according to claim 7, wherein the artificial intelligence entity is further designed to identify the optimized control point in accordance with operational planning.

11. The system according to claim 1, further comprising at least one force sensor designed to record information about the force exerted by the robotically controllable instrument and to transmit the recorded information to the identification device.

12. A computer-implemented method for dynamically optimizing the control point of at least one robotically controllable instrument, comprising at least the following steps:
continuously recording information relating to the position and a control point of at least one robotically controllable instrument;
identifying at least one optimized control point on the basis of the information relating to the position and the control point of the at least one robotically controllable instrument; and
transmitting motion commands to a robot that controls the at least one robotically controllable instrument, wherein the motion commands are designed to instruct the robot to control the at least one robotically controllable instrument from one of the at least one optimized control points.

13. A master-slave system for carrying out medical procedures, at least comprising:
a robotically controllable instrument;
a robot designed to control the robotically controllable instrument from a control point; and
a system according to claim 1,
wherein the system is configured to identify at least one optimized control point and to transmit motion commands to the robot, wherein the motion commands are designed to instruct the robot to control the at least one robotically controllable instrument from one of the at least one optimized control points.
